(19) United States
(12) Patent Application Publication
(10) Pub. No.: US 2010/0179104 A1
(43) Pub. Date: Jul. 15, 2010

(54) SYNTHETIC NUTRITIONAL FORMULATIONS

(76) Inventor: Willem Wassenaar, Toronto (CA)

Correspondence Address: CLARK & BRODY, 1700 Diagonal Road, Suite 510, Alexandria, VA, 22314, US

(21) Appl. No.: 12/730,413

(22) Filed: Mar. 24, 2010

Related U.S. Application Data
(62) Division of application No. 11/512,593, filed on Aug. 30, 2006.
(60) Provisional application No. 60/713,469, filed on Sep. 1, 2005.

(51) Int. Cl. A61K 31/7008; A61K 31/7004; A61P 37/04; A61P 17/02

(52) U.S. Cl. 514/62; 514/23

(57) ABSTRACT

The present invention provides a synthetic nutritional formulation comprising a substantially pure free N-acetyl glucosamine in concentrations effective for enhancing organ development, enhancing an immune response, or increasing tissue repair or healing in a subject. A synthetic nutritional formulation of the invention may optionally comprise one or both of substantially pure free sialic acid and/or substantially pure free fucose.

This application claims priority to U.S. application Ser. No. 60/713,469 filed Sep. 1, 2005.

### FIELD OF THE INVENTION

The invention relates generally to synthetic nutritional formulations and methods using the formulations.

### BACKGROUND OF THE INVENTION

Free sugars including N-acetyl-D-glucosamine (GlcNAc), sialic acid (N-acetylneuraminic acid or NANA), and fucose and are critical components for physiological processes such as organ development and wound healing. Developing and unhealthy individuals often do not have the required types or quantities of enzymes to breakdown oligosaccharides to important free sugars. Compositions and methods that provide critical sugar components for physiological processes such as organ development, the immune response, and wound healing would be of considerable benefit to these individuals.

Human breast milk is unique among mammals in that it is a rich source of many higher oligosaccharides. The oligosaccharide fraction of human breast milk is present at about 18 grams per liter, making it the third largest solute in milk. The oligosaccharides in human breast milk vary from 3 to 11 units in length, and they are characterized by a lactose moiety at their reducing ends and a sialic acid (when present) or a fucose at the non-reducing end. N-acetyl-D-glucosamine is also present in human breast milk oligosaccharides. The concentrations of oligosaccharide bound GlcNAc and sialic acid from full-term human breast milk decreases significantly from weeks 1 to 13 postnatally. Preterm milk contains higher concentrations of each component but temporal changes are similar to those seen in full-term milk (Miller, J. B. and S. Bull, et al, 1994, J. Pediatr Gastroenterol Nutr 19(4):371-6). While human breast milk contains significant amounts of N-acetyl-D-glucosamine, sialic acid and fucose, most infant formulas are deficient in these components. Sanchez-Diaz, A., et al. (1997, J. Pediatr Gastroenterol Nutr., 24(4): 405-10) determined that the total sialic acid and oligosaccharide, glycoprotein, and ganglioside sialic acid content of bovine milk-based formulas is only 36, 28, 50, and 20%, respectively, of the amount found in human colostrums or transitional milk.

Special formulations are required for parenteral nutrition which involves the administration of all or part of a patient's nutritional requirements through an intravenous port of entry bypassing the patient's digestive system. Partial parenteral nutrition can be used in patients after a surgical or medical procedure when they are unable to ingest their nutritional requirements by mouth or through a gastric tube. Total parenteral nutrition (TPN) supplies all of a patient's daily nutritional requirements by the intravenous route for short periods of time during hospitalization or for longer periods, for example, in the case of a patient who has lost small-bowel function. TPN is also provided to severely malnourished patients before and after surgery, radiation therapy, or chemotherapy to improve and maintain their nutritional status. In addition, TPN reduces morbidity and mortality, promotes tissue repair, and enhances the immune response when administered to patients having major surgery, or with severe burns and multiple fractures, particularly in the presence of sepsis.

A standard TPN formulation comprises water (30 to 40 mL/kg/day), energy (30 to 60 kcal/kg/day depending on the degree of energy expenditure), and amino acids (1 to 3 g/kg/day depending on the degree of catabolism). Other components include vitamins and minerals. Formulations have been developed for particular indications. For example, some formulations have a reduced amino acid content (e.g., for patients with renal or hepatic failure), while others have non-protein calories provided as a lipid emulsion (e.g. respiratory failure patients). Children who need TPN also have special nutritional requirements. (See, for example, The Merck Manual of Diagnosis and Therapy, M. H. Beers and R. Berkow, Ed., Section 1. Nutritional Disorders Chapter 1, Nutrition: General Considerations).

### SUMMARY OF THE INVENTION

The present invention provides a synthetic nutritional formulation comprising a substantially pure free N-acetyl glucosamine and/or substantially pure free fucose in concentrations effective for enhancing organ development, enhancing immune responses, or increasing tissue repair or healing in a subject. A synthetic nutritional formulation of the invention may optionally comprise substantially pure free sialic acid.

The invention also provides a pharmaceutical composition, comprising a substantially pure free N-acetyl glucosamine and/or substantially pure free fucose, and optionally substantially pure free sialic acid that provides beneficial effects, in particular sustained beneficial effects, following administration or treatment.

The invention additionally provides a method of preparing a nutritional formulation comprising an N-acetyl glucosamine and/or fucose, optionally free sialic acid, and optionally an acceptable carrier.

The invention further provides a method of preparing a stable pharmaceutical composition comprising a substantially pure free N-acetyl glucosamine and/or substantially pure free fucose, and optionally substantially pure free sialic acid adapted to provide beneficial effects, preferably sustained beneficial effects, following treatment.

The invention contemplates the use of a substantially pure free N-acetyl glucosamine and/or fucose, optionally free sialic acid, a synthetic nutritional formulation or composition of the invention in the preparation of a medicament or dietary supplement for preventing and/or treating a disorder requiring enhancement of tissue repair, enhancement of an immune response, and/or organ development.

The invention provides a method for enhancing organ development or an immune response, or increasing tissue repair or healing in a subject comprising administering to the subject a therapeutically effective amount of a substantially pure free N-acetyl glucosamine and/or substantially pure free fucose, and optionally substantially pure free sialic acid to provide beneficial effects.

The invention also provides a kit comprising a substantially pure free N-acetyl glucosamine and/or substantially pure free fucose, and optionally substantially pure free sialic acid, or a composition of the invention.

### Glossary

Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.” The term “about” means plus or minus 0.1 to 50%, 5-50%, or 10-40%, preferably 10-20%, more preferably 10% or 15%, of the number to which reference is being made. Further, it is to be understood that “a,” “an,” and “the” include plural referents unless the content clearly dictates otherwise. Thus, for example, reference to a composition containing “a compound” includes a mixture of two or more compounds.

All percentages, parts and ratios as used herein are by weight of the total formulation or composition, unless otherwise specified. All such weights as they pertain to listed ingredients are based upon the active level and therefore do not include by-products that may be included in commercially available materials, unless otherwise specified.

All combinations of method or process steps as used herein can be performed in any order, unless otherwise specified or clearly implied to the contrary by the context in which the recited combination is made.

The formulations, compositions, and methods of the invention may comprise, consist of, or consist essentially of the elements and limitations of the invention described herein, as well as any additional or optional ingredients, components of limitations described herein or otherwise useful in the applications disclosed herein.

The terms “subject”, “individual”, or “patient” refer to an animal including a warm-blooded animal such as a mammal. Mammal includes without limitation any members of the Mammalia. In general, the terms refer to a human. The terms also include domestic animals bred for food or as pets. In aspects of the invention the subject is a “newborn infant”. A “newborn infant” refers to an infant less than about 3 months of age including an infant from zero to about 4 weeks of age. A “preterm infant” refers to infants born before 37 weeks of a pregnancy.

The term “infant formula” means a nutritional composition designed for infants which contains suitable proteins, lipids, vitamins, carbohydrates, and minerals to potentially serve as the sole source of nutrition when provided in sufficient quantities. The term refers to synthetic nutritional formulas and therefore specifically excludes human milk, cow's milk, or any other natural whole milk product. The amount of lipids/fats, proteins, and carbohydrates typically can vary from about 3 to about 7 g/100 kcal, 1 to about 5 g/100 kcal, and 6 to about 16 g/100 kcal, respectively. Represented another way, the protein may comprise from about 4 to about 40% of the total calories, including from about 10 to about 30%, also including from about 15 to about 25%; the carbohydrate may comprise less than 40% of the total calories, including from about 5 to about 37%, also including less than about 36%, and also including from about 20 to about 33%; and the fat/lipid may comprise the remainder of the formula calories, typically less than about 60% of the calories, including from about 30 to about 60%.

When diluted or reconstituted, if initially in concentrate or powder form, to the ready to feed state, a typical infant formula can contain about 10-35 g/L of protein, 20-50 g/L of lipid; 60-110 g/L of carbohydrates and other various components such as vitamins, minerals, fibre, emulsifiers, and the like.

The term “infant formula” includes so-called “pre-term infant” and “term” formulas well known to those skilled in the art. Minimum and maximum nutrient requirements for infant formulas are prescribed by national agencies such as the Food and Drug Administration.

Many different sources and types of lipids, carbohydrates, and proteins are known and can be used in infant formulas for the present invention, provided that such nutrients are compatible with the synthetic nutritional formulations of the present invention, and are safe and effective for their intended use, and do not otherwise unduly impair product performance

Sources of lipids for use in infant formula can be any used in the art, including without limitation vegetable oils such as palm oil, soybean oil, palm olein oil, corn oil, canola oil, coconut oil, medium chain triglyceride oils, high oleic sunflower oil, and high oleic safflower oil. Carbohydrate sources can be any used in the art, including without limitation lactose, glucose polymers, corn syrup solids, maltodextrins, sucrose, starch, and rice syrup solids. Suitable protein sources can be any used in the art, and include without limitation whey protein, casein, soy protein, hydrolyzed protein, non-hydrolyzed protein, and amino acids, and combinations thereof.

Examples of infant formulas and methods for their preparation are described, for example in U.S. Pat. No. 6,146,670 to Pieto et al.; U.S. Pat. No. 5,492,899 to Masor et al.; U.S. Pat. No. 5,021,245 to Borschel et al.; U.S. Pat. No. 5,602,109 to Masor et al.; U.S. Pat. No. 4,670,268 to Mahmoud; and U.S. Pat. No. 4,216,236 to Mueller et al.

Exemplary infant formulas which are commercially available include Enfamil, Prosobee, Pregestimil, Portagen, Nutramigen, Lofenalac, Lactofree, Gerber, Alacta, O-Lac, Prosolac (Mead Johnson & Company, Evansville, Ind.), Similac, Isomil (Ross Laboratories, Columbus, Ohio), SMA, Nursoy, Wysoy, Infasoy, Bonna Mayorcitos, Starmil, (Wyeth Laboratories, Philadelphia, Pa.), Alprem, Soylac, Follow-Up, Goodstart (Nestle Carnation), Nenatal, Prematalac, Ammiron, Nutrilon, Nutri-Soja, Farilon, Cow & Gate, Camelpow, Nenatal, Pepti-Jr (Nutricia/Cow & Gate, Netherlands), and Preaptamil, Aptamil, Milumil, Lemiel, Nektarmil, HN-25, GES-45, SOM, Pregomin (Milupa, Germany). A particular infant formula contains the ingredients set out in the table below:

| Nutrient | Unit | Amount per 1 litre |
|---|---|---|
| Calories | kcal | 670 |
| Protein | grams | 14.7 |
| Fat | grams | 34.2 |
| Total | grams | 75 |
| Carbohydrate | | |
| Linoleic Acid | | |
| Linolenic Acid | | |

| | |
|---|---|
| Vitamins | As prescribed by regulation |
| | and nutritional best practices |
| Minerals | As prescribed by regulation |
| | and nutritional best practices |
| Other Components | As prescribed nutritional best |
| | practices |

A “dietary formula” refers to a product for enteral, parenteral, or oral feeding to patients that require supplemental or total nutritional support. Some dietary formulas are designed as supplements and some are designed for total nutrition. A typical formula consists of the following ingredients: water, sugar (sucrose), corn syrup, maltodextrin (corn), calcium caseinate, high-oleic safflower oil, canola oil, soy protein isolate, whey protein concentrate, corn oil, calcium phosphate tribasic, potassium citrate, magnesium phosphate dibasic, natural and artificial flavor, soy lecithin, sodium citrate, magnesium chloride, salt (sodium chloride), carrageenan, choline chloride, potassium chloride, ascorbic acid, ferrous sulfate, alpha-tocopheryl acetate, zinc sulfate, niacinamide, calcium pantothenate, manganese sulfate, cupric sulfate, vitamin A palmitate, thiamine chloride hydrochloride, pyridoxine hydrochloride, riboflavin, folic acid, chromium chloride, biotin, sodium molybdate, sodium selenate, potassium iodide, phylloquinone, vitamin D3 and cyanocobalamin. Examples of commercially available dietary formula are listed in Table 4 below.

The typical usual carbohydrates used in dietary formula include corn syrup solids, sucrose, fructooligosaccharides, maltodextrin, lactose, glucose, and other complex carbohydrates. The quantities of these carbohydrates vary with the product and the presentation. Typically in a dietary product 40% to 60% of the calories are supplied by carbohydrates. However this may vary with the purpose of the dietary product. A product such as MCT Oil® (Novartis) has no carbohydrates and no protein. RCF® Ross carbohydrate free soy formula is carbohydrate free and is intended for persons who are unable to tolerate the type or amount of carbohydrate in milk or persons suffering from seizure disorders requiring a ketogenic diet. Other products such as Tolerex® Powder (Novartis) are fat free products in which 91% of the calories are supplied by carbohydrates.

Dietary formula also includes products that exclude components that patients with certain diseases or severity of illness do not have the enzymatic machinery to break down into their elemental parts. An example of such a formula is Tolerex® (Novartis) which contains amino acids but no proteins.

A standard total parenteral formula comprises water (30 to 40 mL/kg/day), energy (30 to 60 kcal/kg/day depending on the degree of energy expenditure), amino acids (1 to 3 g/kg/day depending on the degree of catabolism), vitamins and minerals. Glucose containing solutions used in total parenteral nutrition can vary in concentration from 5% weight/volume to 70% weight/volume. A formula is generally chosen having regard to a patient's fluid requirements relative to caloric requirements. Higher concentrations are used in patients that require that volume intake be limited. A typical 5% glucose solution contains 5 grams of glucose per 100 mL of water. Some formulations are adapted for particular patients, and for example, have reduced amino acid content (e.g., for patients with renal or hepatic failure), non-protein calories provided as a lipid emulsion (e.g. respiratory failure patients), or special nutritional requirements (e.g. for children). Examplary parenteral formula include Oliclinomel®, Clinoleic®, and Ivelip® (Baxter), Kabiven®, Intralipid®, and Dipetiven® (Fresenius Kabi). The products may be provided in containers with lipid emulsions and aqueous solutions of glucose and electrolytes or as multi-chamber bags containing each of the solutions.

Dietary formula useful as acceptable carriers for enteral and oral feeding formulations are set out in Table 4 below.

The term “pharmaceutically acceptable carrier, excipient, or vehicle” refers to a medium which does not interfere with the effectiveness or activity of an active ingredient and which is not toxic to the hosts to which it is administered. A carrier, excipient, or vehicle includes diluents, binders, adhesives, lubricants, disintegrates, bulking agents, wetting or emulsifying agents, pH buffering agents, and miscellaneous materials such as absorbants that may be needed in order to prepare a particular composition. Examples of carriers, excipients, and vehicles include but are not limited to saline, buffered saline, water, glycerol, ethanol, and combinations thereof. The use of such media and agents for an active substance is well known in the art.

A “beneficial effect” refers to an effect of an N-acetyl glucosamine, fucose, and/or sialic acid or composition thereof including favorable nutritional, pharmacological and/or therapeutic effects. The beneficial effect may be a statistically significant effect in terms of statistical analysis of an effect of a substantially pure free N-acetyl glucosamine and/or substantially pure free fucose, and optionally substantially pure free sialic acid versus the effects without the compound(s). “Statistically significant” or “significantly different” effects or levels may represent levels that are higher or lower than a standard. In embodiments of the invention, the difference may be 1.5, 2, 3, 4, 5, or 6 times higher or lower compared with the effect obtained without a substantially pure free N-acetyl glucosamine, and optionally substantially pure free fucose and substantially pure free sialic acid. In aspects of the invention the beneficial effect is characterized by one or more of the following:
a) Enhancement of organ development in an infant, in particular a newborn infant, in particular, enhancement of development of the brain, liver, kidneys.
b) Processing of glucose more efficiently for energy production and thereby accelerating growth and development.
c) Enhancement of wound healing and tissue repair, in particular, enhancement of mobility of fibroblasts and leukocytes to injured sites, and increasing the availability of glucose which is a source of energy and structural molecules for organ and tissue repair.
d) Enhancement of an immune response, in particular enhancing humoral and/or cellular immunity,

“Therapeutically effective amount” relates to the amount or dose of a substantially pure free N-acetyl glucosamine, fucose, and/or sialic acid, or composition of the invention that will lead to one or more desired effect, in particular a beneficial effect, more particularly one or more sustained beneficial effects. A therapeutically effective amount of a substance can vary according to factors such as the disease state, age, sex, and weight of the individual, and the ability of the substance to elicit a desired response in the individual. Dosage regimens may be adjusted to provide the optimum nutritional or therapeutic response (e.g. sustained beneficial effects). For example, several divided doses may be administered daily or the dose may be proportionally reduced as indicated by the exigencies of the therapeutic situation.

An “N-acetyl glucosamine” refers to N-acetyl-D-glucosamine compound, pharmaceutically acceptable salts, isomers, and functional derivatives thereof. An N-acetyl glucosamine can be prepared using conventional processes or it may be obtained from commercial sources. For example, U.S. Pat. No. 6,693,188 describes the preparation of N-acetyl-D-glucosamine from microbial biomass.

“Fucose” refers to a L-fucose compound, pharmaceutically acceptable salts, isomers, and functional derivatives thereof. “Sialic acid” refers to sialic acid compound, pharmaceutically acceptable salts, isomers, and functional derivatives thereof. Fucose and sialic acid can be prepared using conventional processes or they may be obtained from commercial sources

“Pharmaceutically acceptable salt(s),” means a salt that is pharmaceutically acceptable and has the desired pharmacological properties. Suitable salts include salts that may be formed where acidic protons in the compounds are capable of reacting with inorganic or organic bases. Suitable inorganic salts include those formed with alkali Metals, e.g. sodium and potassium, magnesium, calcium, and aluminum. Suitable organic salts include those formed with organic bases such as the amine bases, e.g. ethanolamine, diethanolamine, triethanolamine, tromethamine, N-methylglucamine, and the like. Suitable salts also include acid addition salts formed with inorganic acids (e.g. hydrochloride and hydrobromic acids) and organic acids (e.g. acetic acid, citric acid, maleic acid, and the alkane- and arene-sulfonic acids such as methanesulfonic acid and benzenesulfonic acid).

A “functional derivative” refers to a compound that possesses a biological activity (either functional or structural) that is substantially similar to the biological activity of an N-acetyl glucosamine, fucose, or sialic acid. The term “functional derivative” is intended to include “variants” “analogs” or “chemical derivatives” of N-acetyl glucosamine, fucose or sialic acid. The term “variant” is meant to refer to a molecule substantially similar in structure and function to N-acetyl glucosamine, fucose, or sialic acid, or a part thereof. A molecule is “substantially similar” to N-acetyl glucosamine, fucose, or sialic acid if both molecules have substantially similar structures or if both molecules possess similar biological activity. The term “analog” refers to a molecule substantially similar in function to an N-acetyl glucosamine, fucose, or sialic acid. The term “chemical derivative” describes a molecule that contains additional chemical moieties which are not normally a part of the base molecule.

Formulations, compositions, and methods of the invention utilize N-acetyl glucosamine, fucose and sialic acid compounds that are at least substantially pure. In general “pure” means better than 85%, 86%, 87%, 88%, 89%, 90%, 91%, 92%, 93%, 94%, 95%, 96%, 97%, 98%, or 99% pure, and “substantially pure” means a compound available for incorporation into a formulation or composition, that has only those impurities that can not readily nor reasonably be removed by conventional purification processes.

“Free” means free of other molecules including without limitation proteins, lipids (fats) and carbohydrates.

A compound utilized in a formulation, composition, or method of the invention can contain one or more asymmetric centers and may give rise to enantiomers, diasteriomers, and other stereoisomeric forms which may be defined in terms of absolute stereochemistry as (R)- or (S)-. Therefore, the invention includes all such possible diasteriomers and enantiomers as well as their racemic and optically pure forms. Optically active (R)- and (S)-isomers may be prepared using chiral synthons or chiral reagents, or resolved using conventional techniques. When a compound described herein contains centers of geometric asymmetry, and unless specified otherwise, it is intended that the compounds include both E and A geometric isomers. All tautomeric forms are intended to be included within the scope of the invention.

### DETAILED DESCRIPTION OF EMBODIMENTS

Reference now will be made in detail to the embodiments of the invention, illustrated by one or more examples set forth below.

### Formulations

Innovative aspects of the present invention involve the use of substantially pure free N-acetyl glucosamine and optionally substantially pure free fucose and substantially pure free sialic acid to provide nutritional formulations with enhanced levels of these specific monosaccharide components that are desirable for organ development, immune responses, and/or wound healing. These formulations are particularly suitable in infants and very ill patients that do not have the enzymatic machinery to break down oligosaccharides. Typically dietary sources of these sugars in the form of glycosaminoglycans, glycolipids and glycoproteins must be broken down by enzymes (in particular, N-acetyl-beta-hexosaminidases, fucosidase and sialidases) to individual sugars before they can be used. A developing organism does not process a full complement of enzymes at birth. In addition, the demand may be so high that the neonate is not able to process these complex structures fast enough because the digestive system and liver are not fully developed and thus are not able to meet the requirements for N-acetyl glucosamine, fucose, and/or sialic acid. Similarly in times of severe illness the demand for these sugars may also be very high and a patient may not be able to obtain a sufficient amount of free or monomeric N-acetyl glucosamine, fucose and/or sialic acid from processing complex nutritional sources. If the illness is severe a patient may be given a dietary formula e.g., a commercial meal replacement or parenteral nutritional preparation. While these preparations may contain oligosaccharides the patient may not have the digestive enzymes to break down the oligosaccharides. Similarly, if a patient is fed by the intravenous route there is no dietary source of free or monomeric L-fucose, N-acetyl -D-glucosamine, or sialic acid.

This invention is directed to a synthetic nutritional formulation comprising a substantially pure free N-acetyl glucosamine in concentrations effective for enhancing organ development, enhancing immune responses, or increasing tissue repair or healing in a subject. A synthetic nutritional formulation of the invention may optionally comprise substantially pure free fucose and/or substantially pure free sialic acid.

In an aspect, the invention provides a synthetic nutritional formulation for mammalian consumption and particularly human consumption for the purpose of enhancing organ development, an immune response or wound healing comprising a substantially pure free N-acetyl glucosamine and optionally substantially pure free fucose and/or substantially pure free sialic acid. In aspects of the invention, the formulation comprises a substantially pure free N-acetyl glucosamine and a substantially pure free fucose. In other aspects, the invention provides a formulation comprising a substantially pure free N-acetyl glucosamine, a substantially pure free fucose, and a substantially pure free sialic acid.

A synthetic nutritional formulation of the invention is preferably pleasant tasting and provides beneficial effects.

The invention provides a synthetic nutritional formulation comprising an enhanced concentration of substantially pure free N-acetyl glucosamine, and optionally enhanced concentration of substantially pure free fucose and/or a substantially pure free sialic acid.

In embodiments of the invention, a synthetic nutritional formulation comprises 50 to 10,000 mg, 300 to 5000, 300 to 2000, or 300 to 1500 mg of N-acetyl glucosamine.

In aspects of the invention, a synthetic nutritional formulation is in the form of a liquid or powder, in particular an elixir, suspension, emulsion, solution, syrup, or powder. In a particular aspect, a synthetic nutritional formulation is formulated as a beverage, but may be formulated in granule or powder form.

The invention still further comprises a nutritional formulation that is a beverage comprising a fluid, and an ingestible amount of a substantially pure free N-acetyl glucosamine, and optionally substantially pure free fucose and/or substantially pure free sialic acid, for enhancing organ development or an immune response, or increasing tissue repair or healing in a subject.

A synthetic nutritional formulation of the invention may be adapted for use in combination with another formula.

In an aspect, a synthetic nutritional formulation of the invention may be adapted for use in combination with an infant formula. Thus, in an aspect, the invention provides a synthetic nutritional formulation comprising a substantially pure free N-acetyl glucosamine, wherein the concentration of N-acetyl glucosamine is similar to the concentration of N-acetyl glucosamine in breast milk, in particular human breast milk.

In an embodiment the invention provides a synthetic nutritional formulation comprising free N-acetyl glucosamine and free fucose, wherein the concentrations of N-acetyl glucosamine and fucose are similar to the concentrations of N-acetyl glucosamine and fucose in breast milk, in particular human breast milk

In another embodiment the invention provides a synthetic nutritional formulation comprising free N-acetyl glucosamine, free sialic acid and free fucose, wherein the concentrations of free N-acetyl glucosamine, free sialic acid, and free fucose are similar to the concentrations of free N-acetyl glucosamine, sialic acid, and fucose in breast milk, in particular human breast milk.

In particular embodiments, the concentrations of N-acetyl glucosamine, sialic acid and/or fucose are similar to, or about the same as, the concentrations of N-acetyl glucosamine, sialic acid and/or fucose shown in the following Table:

| | Human Breast Milk | Human Breast Milk At |
|---|---|---|
| | At 1 Week | 13 Weeks |
| Monosaccharide | mg/L (mean +/− SD) | mg/L (mean +/− SD) |
| N-acetyl-D-glucosamine | 1,459 +/− 282 | 646 +/− 214 |
| Sialic acid | 879 +/− 157 | 256 +/− 82 |
| Fucose | 660 +/− 192 | 432 +/− 180 |

In a further embodiment the invention provides a synthetic nutritional formulation having enhanced amounts of free N-acetyl glucosamine, and optionally free fucose and/or free sialic acid, compared with the amounts of N-acetyl glucosamine, and optionally fucose and/or sialic acid in breast milk, in particular human breast milk. In particular embodiments, the amounts of free N-acetyl glucosamine, and optionally free fucose and/or free sialic acid are greater than the amounts in the above Table.

A synthetic nutritional formulation of the invention may be adapted for use in combination with a dietary formula. In an aspect, the invention provides a synthetic nutritional formulation comprising a substantially pure free N-acetyl glucosamine, and optionally free fucose and/or free sialic acid, wherein the concentration of free N-acetyl glucosamine, fucose and/or sialic acid are similar to the concentrations obtained from processing of oligosaccharides in complex nutritional sources.

A synthetic nutritional formulation may additionally comprise an acceptable carrier. In embodiments of the invention an acceptable carrier is water, a processed milk product, soy milk, an infant formula, dietary formula, parenteral nutrition preparation, an electrolyte solution, an aqueous phase of fat emulsions, or a pharmaceutically acceptable carrier, excipient, or vehicle.

In a particular aspect the synthetic nutritional formulation comprises a carrier which is an infant formula. Thus, the invention provides an infant formulation comprising as an additive thereto a substantially pure free N-acetyl glucosamine, the formulation being essentially free of N-acetyl glucosamine prior to addition of the N-acetyl glucosamine thereto. The invention also provides an infant formulation comprising as an additive thereto a substantially pure free fucose, the formulation being essentially free of fucose prior to addition of the fucose thereto. An infant formulation of the invention may optionally comprise substantially pure free sialic acid.

In an embodiment, the invention provides an infant formulation comprising enhanced amounts of a free N-acetyl glucosamine and optionally enhanced amounts of free fucose and/or free sialic acid, and in particular an infant formulation that has nutritionally enhanced amounts of free N-acetyl glucosamine, and optionally nutritionally enhanced amounts of free fucose and/or free sialic acid while also providing a lipid, protein, and carbohydrate profile that is similar to breast milk.

In another embodiment, the invention provides a pharmaceutical or nutritional or food composition for feeding infants comprising an infant formula base formulated to provide at least a source of calories and protein, and as a supplement a nutritional formulation of the invention.

In an aspect of an infant formulation of the invention, the levels of a substantially pure free N-acetyl glucosamine, substantially pure free fucose, and/or substantially pure free sialic acid mimic the levels of the component(s) in breast milk, in particular the levels over the period 0 to 2 years, more particularly 0 to 12 months, of lactation.

In an embodiment of an infant formulation of the invention, the concentration of a substantially pure free N-acetyl glucosamine is about 50 to 2500, 50 to 2000, 50 to 1500, 50 to 100, 200 to 1500, 500 to 1200, 600 to 1600, or 800 to 1500 mg N-acetyl glucosamine per liter of formulation.

In particular embodiments of an infant formulation of the invention, the concentration of a substantially pure free N-acetyl glucosamine is between about 0.2 to 2.5 g/L, 0.4 to 2 g/L, 0.4 to 1.5 g/L; 0.3-2.5 g/L, 0.2 to 2.5 g/L or 0.4 to 1.8 g/L; 0.03-4 wt %, 0.5 to 2.4 wt %, 0.04-2 wt %, 0.04-1 wt %, 0.04-0.5 wt %, or 0.04-0.18 wt %; or 0.5 to 3% or 0.5 to 2.5%, more particularly 0.53-2.4% of the total carbohydrates.

In other embodiments of the invention an enhanced substantially pure free N-acetyl glucosamine product with high N-acetyl glucosamine content (e.g. above 600, 1000, or 1500 mg per liter) may be utilized.

In embodiments of an infant formulation of the invention, the concentration of a substantially pure free fucose is about 20 to 1000, 100 to 1000, 200 to 1000, 200 to 800, 250 to 900, 300 to 700, 400 to 700, 400 to 670, or 400 to 600 mg of fucose per liter of formulation. In embodiments of the invention an enhanced substantially pure free fucose product with high fucose content (e.g. above 400 mg per liter) may be utilized.

In particular embodiments of an infant formulation of the invention, the concentration of a substantially pure free fucose is about 0.2 to 1 g/L, 0.25 to 0.9 g/L, 0.25 to 0.6 g/L; 0.02-1.5 wt %, 0.3 to 1.2 wt %, 0.02-1.0 wt %, 0.02-8 wt %, or 0.03-0.09 wt %; or 0.3 to 2% or 0.3 to 1.5%, in particular 0.33-1.20% of the total carbohydrates.

In embodiments of an infant formulation of the invention, the concentration of a substantially pure free sialic acid is about 250 mg (as found in mature human breast milk) to about 1500 mg per liter (as found in human colostrums), and in an embodiment about 300 to about 600 mg per liter. In an embodiment of the invention which is a dry powder product the concentration of sialic acid is about 190-230 mg/gm protein or about 100 to 130 mg/gm powder.

In a particular aspect the synthetic nutritional formulation comprises a carrier which is a dietary formula. Thus, the invention provides a dietary formulation, in particular a parenteral dietary formulation, comprising as an additive thereto a substantially pure free N-acetyl glucosamine, the formula being essentially free of N-acetyl glucosamine prior to addition of the N-acetyl glucosamine thereto. The invention also provides a dietary formulation comprising as an additive thereto a substantially pure free N-acetyl glucosamine and/or substantially pure free fucose, the formulation being essentially free of N-acetyl glucosamine and/or fucose prior to addition of the N-acetyl glucoamine and/or fucose thereto. A dietary formulation of the invention may optionally comprise substantially pure free sialic acid the formulation being essentially free of sialic acid prior to addition of the sialic acid thereto.

In embodiments of dietary formula of the invention, substantially pure N-acetyl glucosamine and fucose comprise 2-5% or 2-4%, in particular 2-3% by weight of the total carbohydrate present in the formulation.

A dietary formula in combination with a synthetic nutritional formulation or a dietary formulation of the invention are particularly suited for patients with involuntary weight loss due to the effects of cancer or cancer treatments, bowel disease, kidney disease, liver disease, lung disease and recovery from surgery. These products may be a liquid, a pudding, a powder or a solid bar or a cookie. The powders may be reconstituted to prepare a liquid. The formulations may be taken by mouth, through a tube, where the distal opening is placed in the stomach or bowel. The formulations may also be administered intravenously for parenteral nutrition in situations where the patient does not have a functioning digestive tract. These dietary preparations may be used in a wide set of clinical circumstances and for this purpose many variations in formulation are commercially available.

The invention additionally provides a method of preparing a synthetic nutritional formulation, in particular an infant formulation or dietary formulation of the invention comprising mixing a substantially pure free N-acetyl glucosamine, optionally substantially pure free fucose and/or optionally substantially free sialic acid, and optionally an acceptable carrier.

In an aspect, the invention provides methods to make commercially available elixirs, suspensions, emulsions, solutions, syrups, and powders which contain an N-acetyl glucosamine, optionally free fucose or free sialic acid. In another aspect, a synthetic nutritional formulation is formulated as a beverage, but may be formulated in granule or powder form. Elixirs, suspensions, emulsions, beverages, etc. may be prepared by methods known in the art.

### Pharmaceutical Compositions

The invention also provides a pharmaceutical composition, comprising a substantially pure free N-acetyl glucosamine and/or free fucose, and optionally free sialic acid that provides beneficial effects, in particular sustained beneficial effects, following treatment. The beneficial effects provided by a composition of the invention can include enhanced therapeutic effects. The invention also provides a pharmaceutical composition intended for administration to a subject to provide beneficial effects, in particular sustained beneficial effects, comprising substantially pure free N-acetyl glucosamine and/or substantially pure free fucose, optionally substantially pure free sialic acid, optionally together with a pharmaceutically acceptable carrier, excipient, or vehicle.

Pharmaceutical compositions of the invention can be selected that have sustained beneficial effects (e.g., sustained therapeutic effects), preferably statistically significant sustained beneficial effects. In an embodiment, a pharmaceutical composition with statistically significant sustained beneficial effects is provided comprising a therapeutically effective amount of a substantially pure free N-acetyl glucosamine.

The invention also provides a pharmaceutical composition for enhancing organ development in an infant, newborn infant, or preterm infant comprising a therapeutically effective amount of a substantially pure free N-acetyl glucosamine and for fucose, optionally free sialic acid, to provide a sustained beneficial effect, in a pharmaceutically acceptable carrier, excipient, or vehicle. The compositions of the present invention or fractions thereof typically comprise suitable pharmaceutically acceptable carriers, excipients, and vehicles selected based on the intended form of administration, and consistent with conventional pharmaceutical practices.

Suitable pharmaceutical carriers, excipients, and vehicles are described in the standard text, Remington's Pharmaceutical Sciences, Mack Publishing Company. By way of example for oral administration in the form of a capsule or tablet, the active components can be combined with an oral, non-toxic pharmaceutically acceptable inert carrier such as lactose, starch, sucrose, methyl cellulose, magnesium stearate, glucose, calcium sulfate, dicalcium phosphate, mannitol, sorbital, and the like. For oral administration in a liquid form, the drug components may be combined with any oral, non-toxic, pharmaceutically acceptable inert carrier such as ethanol, glycerol, water, and the like. Suitable binders (e.g. gelatin, starch, corn sweeteners, natural and synthetic gums, and waxes), lubricants (e.g. sodium oleate, sodium stearate, magnesium stearate, sodium benzoate, sodium acetate, and sodium chloride), disintegrating agents (e.g. starch, methyl cellulose, agar, bentonite, and xanthan gum), flavoring agents, and coloring agents may also be combined in the compositions or components thereof. Compositions as described herein can further comprise wetting or emulsifying agents, or pH buffering agents.

A pharmaceutical composition can be a liquid solution, suspension, emulsion, tablet, pill, capsule, sustained release formulation, or powder. The compositions can be formulated as a suppository, with traditional binders and carriers such as triglycerides. Oral formulations can include standard carriers such as pharmaceutical grades of mannitol, lactose, starch, magnesium stearate, sodium saccharine, cellulose, magnesium carbonate, etc. Various delivery systems are known and can be used to administer a composition of the invention, e.g. encapsulation in liposomes, microparticles, microcapsules, and the like.

Compositions for parenteral administration may include sterile aqueous or non-aqueous solvents, such as water, isotonic saline, isotonic glucose solution, buffer solution, or other solvents conveniently used for parenteral administration of therapeutically active agents. Compositions for parenteral administration may alternatively or additionally include syrups, aqueous or oil suspensions and emulsions with edible oil such as cottonseed oil, coconut oil or peanut oil. Dispersing or suspending agents that can be used for aqueous suspensions include synthetic or natural gums, such as tragacanth, alginate, acacia, dextran, sodium carboxymethylcellulose, gelatin, methylcellulose, and polyvinylpyrrolidone. A composition intended for parenteral administration may also include conventional additives such as stabilizers, buffers, or preservatives, e.g. antioxidants such as methylhydroxybenzoate or similar additives.

In an embodiment, a composition herein is formulated in accordance with routine procedures as a pharmaceutical composition adapted for subcutaneous or intravenous administration to human beings to provide a beneficial effect, in particular a sustained beneficial effect. Typically, compositions for subcutaneous or intravenous administration are solutions in sterile isotonic aqueous buffer. Where necessary, the composition may also include a solubilizing agent and a local anesthetic to ameliorate pain at the site of the injection. Generally, the ingredients are supplied either separately or mixed together in unit dosage form, for example, as a dry, lyophilized powder or water-free concentrate in a hermetically sealed container such as an ampoule or sachette, for example, indicating the quantity of active agent. Where the composition is to be administered by infusion, an ampoule of sterile water or saline for injection can be provided so that the ingredients may be mixed prior to administration.

Compositions of the invention can be formulated as pharmaceutically acceptable salts.

In an embodiment, the invention relates to a liquid drug formulation comprising pharmaceutically acceptable salts of an N-acetyl glucosamine, and to lyophilized drug formulations that can be reconstituted to provide suspensions that are stable and suitable for parenteral administration.

In a particular embodiment, the invention relates to an aqueous composition comprising pharmaceutically acceptable salts of an N-acetyl glucosamine, and a solvent system which effects solubilization. The invention also provides a drug comprising an aqueous formulation of pharmaceutically acceptable salts of an N-acetyl glucosamine with at least one solubilizer.

A composition of the invention may be sterilized by, for example, filtration through a bacteria retaining filter, addition of sterilizing agents to the composition, irradiation of the composition, or heating the composition. Alternatively, the compounds or compositions of the present invention may be provided as sterile solid preparations e.g. lyophilized powder, which are readily dissolved in sterile solvent immediately prior to use.

In addition to the formulations described herein, the compositions can also be formulated as a depot preparation. Such long acting formulations may be administered by implantation (for example, subcutaneously or intramuscularly) or by intramuscular injection. Thus, for example, the fractions may be formulated with suitable polymeric or hydrophobic materials (for example, as an emulsion in an acceptable oil), or ion exchange resins, or as sparingly soluble derivatives, for example, as a sparingly soluble salt.

The invention further provides a method of preparing a stable pharmaceutical composition comprising a substantially free N-acetyl glucosamine and/or fucose, optionally free sialic acid adapted to provide beneficial effects, preferably sustained beneficial effects, following treatment. After compositions have been prepared, they can be placed in an appropriate container and labelled for treatment of an indicated condition. For administration of a composition of the invention, such labelling would include amount, frequency, and method of administration.

### Applications

The invention also contemplates the use of a composition comprising a substantially pure free N-acetyl glucosamine and optionally substantially pure free fucose and/or substantially pure free sialic acid for the preparation of a medicament or dietary supplement for preventing and/or treating a disorder requiring enhancement of tissue repair, enhancement of an immune response, and/or organ development. The invention additionally provides uses of a pharmaceutical composition of the invention in the preparation of a medicament or dietary supplement for enhancing organ development or an immune response, or increasing tissue repair or healing in a subject. A medicament or dietary supplement may provide beneficial effects, preferably sustained beneficial effects following treatment.

The invention provides a method for enhancing organ development or an immune response, or increasing tissue repair or healing in a subject comprising administering to the subject a therapeutically effective amount of a free N-acetyl glucosamine and optionally free fucose and/or free sialic acid to provide beneficial effects. In an aspect the invention provides a treatment which results in sustained beneficial effects following treatment.

The invention also contemplates a method for increasing tissue repair, an immune response or healing in an infant subject, in particular a newborn infant or preterm infant, comprising administering to the infant subject an effective amount of a synthetic nutritional formulation comprising a substantially pure free N-acetyl glucosamine and optionally substantially pure free fucose and/or substantially pure free sialic acid, or a composition of the invention.

The invention is directed to a method of enhancing organ development or an immune response, or increasing tissue repair or healing in a human subject, in particular an infant, more particularly a newborn infant or a preterm infant, comprising feeding the subject a therapeutically effective amount of a formulation comprising a substantially pure N-acetyl glucosamine on a regular basis. The pure N-acetyl glucosamine can be fed to the human subject on a daily basis. The method may optionally comprise administering a therapeutically effective amount of substantially pure free fucose and/or substantially pure free sialic acid. The dosage can be about 100 mg to about 10,000 mg of an N-acetyl glucosamine per day, about 100 mg to about 5,000 mg of an N-acetyl glucosamine per day, about 100 mg to about 3,000 mg of N-acetyl glucosamine per day, about 100 mg to about 2,000 mg of N-acetyl glucosamine per day, about 500 mg to about 2,000 mg of N-acetyl glucosamine per day or about 600 mg to about 1500 mg of N-acetyl glucosamine per day. The dosage of fucose can be about 20 mg to about 2500 mg of a fucose per day, about 100 mg to about 2000 mg of fucose per day, about 100 mg to about 1500 mg of fucose per day or about 200 mg to about 1500 mg of fucose per day.

The dosage of sialic acid can be about 50 to about 2000 mg of sialic acid per day, about 100 mg to about 1500 mg of sialic acid per day, about 200 mg to about 1000 mg of sialic acid per day, or about 300 mg to about 600 mg of sialic acid per day.

The invention also includes a regimen for supplementing a subject's diet by administering a synthetic nutritional formulation of the invention to the subject. The invention further includes a regimen for supplementing a subject's diet by administering to the subject a synthetic nutritional formulation comprising a substantially pure free N-acetyl glucosamine, optionally free fucose and/or free sialic acid or neutraceutically acceptable derivatives thereof.

In an aspect, a regimen for supplementing a subject's diet is provided comprising administering to the subject a synthetic nutritional formulation comprising per liter of supplement about 50 to 2500 mg of a substantially pure free N-acetyl glucosamine, and optionally about 20 mg to about 2500 mg of substantially pure free fucose and/or about 50 mg to about 2000 mg of free sialic acid.

The invention also contemplates the use of a composition comprising an N-acetyl glucosamine for the preparation of a medicament or dietary supplement providing beneficial effects, preferably sustained beneficial effects in treating a disorder or disease.

In an embodiment, the invention relates to the use of a therapeutically effective amount of an N-acetyl glucosamine for preparation of a medicament or dietary supplement for providing beneficial effects, preferably sustained beneficial effects, in treating a disorder or disease.

The invention additionally provides uses of an N-acetyl glucosamine or a pharmaceutical composition of the invention in the preparation of medicaments or dietary supplements for beneficial effects, preferably sustained beneficial effects, in the treatment of disorders and diseases.

A formulation or composition of the invention can be administered to a patient by a route effective to provide a beneficial effect. Exemplary routes of administration include intravenous and oral routes. The active ingredients can be administered simultaneously or sequentially and in any order at different points in time, to provide the desired beneficial effects, in particular sustained beneficial effects. An N-acetyl glucosamine, fucose, or sialic acid, or composition of the invention can be formulated for sustained release, for delivery locally or systemically. It lies within the capability of a skilled practitioner to select a form and route of administration that optimizes the effects of the compositions and treatments of the present invention to provide beneficial effects, in particular sustained beneficial effects.

The formulations, compositions, and components thereof may be administered in oral dosage forms such as tablets, capsules (each of which includes sustained release or timed release formulations), pills, powders, granules, elixirs, tinctures, suspensions, syrups, and emulsions. They may also be administered in intravenous (bolus or infusion), intraperitoneal, subcutaneous, or intramuscular forms, all utilizing dosage forms well known to those of ordinary skill in the pharmaceutical arts. Compositions of the invention may be administered by intranasal route via topical use of suitable intranasal vehicles, or via a transdermal route, for example using conventional transdermal skin patches.

### Kits

The invention provides kits comprising an N-acetyl glucosamine, fucose, and/or sialic acid, a formulation, or a pharmaceutical composition of the invention. The kit is a package which houses a container which contains an N-acetyl glucosamine, fucose, and/or sialic acid, a formulation, or a composition of the invention and also houses instructions for administering the N-acetyl glucosamine, fucose, and/or sialic acid, formulation or composition to a subject.

In an aspect, the invention provides a kit comprising a substantially pure free N-acetyl glucosamine and optionally substantially pure free fucose, and/or substantially pure free sialic acid, or a composition of the invention. In another aspect, the invention provides a kit for enhancing organ development or an immune response, or increasing tissue repair or healing containing a composition comprising a substantially pure free N-acetyl glucosamine and optionally substantially pure free fucose, and/or substantially pure free sialic acid, a container, and instructions for use. The composition of a kit can further comprise a pharmaceutically acceptable carrier, excipient, or vehicle.

In embodiments of the invention, a pharmaceutical pack or kit is provided comprising one or more containers filled with one or more of the ingredients of a formulation or composition of the invention to provide a beneficial effect, in particular a sustained beneficial effect. Associated with such container(s) can be various written materials such as instructions for use, or a notice in the form prescribed by a governmental agency regulating the manufacture, use or sale of pharmaceuticals, biological products, or dietary supplements which notice reflects approval by the agency of manufacture, use, or sale for human administration.

The invention will be described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### EXAMPLES

### Example 1

The following Table 1 illustrates an infant formula of the invention with the additional components of both N-acetyl-D-glucosamine and L-fucose (% of total carbohydrate).

TABLE 1
Infant Formula of the Invention

| Nutrient | Unit | Amount per 1 litre |
|---|---|---|
| Calories | Kcal | 670 |
| Protein | Grams | 14.7 |
| Fat | Grams | 34.2 |
| Total Carbohydrate | Grams | 75 (100%) |
| N-acetyl-D-glucosamine | Grams | 1.5 (2% of total) |
| L-fucose | grams | 0.6 (0.8% of total) |
| Linoleic Acid | | |
| Linolenic Acid | | |

| | |
|---|---|
| Vitamins | As prescribed by regulation and |
| | nutritional best practices |
| Minerals | As prescribed by regulation and |
| | nutritional best practices |
| Other Components | As prescribed nutritional |
| | best practices |

### Example 2

The following is an example of an infant feeding formula with the addition of N-acetyl-D-glucosamine (% of total carbohydrate).

TABLE 2
Infant Formula of the Invention

| Nutrient | Unit | Amount per 1 litre |
|---|---|---|
| Calories | Kcal | 670 |
| Protein | Grams | 14.7 |
| Fat | Grams | 34.2 |
| Total Carbohydrate | Grams | 75 (100%) |
| N-acetyl-D-glucosamine | Grams | 1.5 (2% of total) |
| L-fucose | Grams | 0 (0% of total) |
| Linoleic Acid | | |
| Linolenic Acid | | |

| | |
|---|---|
| Vitamins | As prescribed by regulation |
| | and nutritional best practices |
| Minerals | As prescribed by regulation |
| | and nutritional best practices |
| Other Components | As prescribed nutritional |
| | best practices |

### Example 3

The following is an example of an infant feeding formula with the addition of L-fucose (% of total carbohydrate).

TABLE 3
Infant Formula of the Invention

| Nutrient | Unit | Amount per 1 litre |
|---|---|---|
| Calories | Kcal | 670 |
| Protein | Grams | 14.7 |
| Fat | Grams | 34.2 |
| Total Carbohydrate | Grams | 75 (100%) |
| N-acetyl-D-glucosamine | grams | 0 (0% of total) |
| L-fucose | grams | 0.6 (0.8% of total) |
| Linoleic Acid | | |
| Linolenic Acid | | |

| | |
|---|---|
| Vitamins | As prescribed by regulation |
| | and nutritional best practices |
| Minerals | As prescribed by regulation |
| | and nutritional best practices |
| Other Components | As prescribed nutritional |
| | best practices |

### Example 4

A synthetic nutritional formulation of the invention can be combined with a commercially available dietary formula listed in Table 4 below to provide an enteral or oral feeding formulation.

TABLE 4
Commercially Available Nutritional Products for Enteral and Oral Feeding

| Trade name | How Administered | Manufactuerer |
|---|---|---|
| Alimentum Liquid | Oral | Abbott Laboratories Ltd |
| Boost Plus Calories Liquid | Oral | Novartis Nutritional Corporation |
| Compleat | oral, tube | Novartis Nutritional Corporation |
| Criticare HN Liquid | oral | Novartis Nutritional Corporation |
| Crucial Diet | oral, tube | Nestle Clinical Nutritional |
| Ensure Plus Liquid | oral | Abbott Laboratories Ltd |
| Ensure with Fibre Liquid | oral | Abbott Laboratories Ltd |
| Glucerna Tube Feeding | tube | Abbott Laboratories Ltd |
| Isocal Liquid | tube | Novartis Nutritional Corporation |
| IsoSource | tube | Novartis Nutritional Corporation |
| Jevity Liquid | tube | Abbott Laboratories Ltd |
| KetoCal 7.2 kcal/g Powder | oral | SHS North America |
| Lipisorb | oral | Novartis Nutritional Corporation |
| MCT Oil | oral | Novartis Nutritional Corporation |
| Modulen IBD | oral | Nestle Clinical Nutritional |
| Neocate Powder | oral | SHS North America |
| Nepro | oral | Abbott Laboratories Ltd |
| NovaSource Renal Liquid | tube | Novartis Nutritional Corporation |
| NuBasic Liquid | oral | Nestle Clinical Nutritional |
| Nutren 1.0 with Fibre Liquid | oral | Nestle Clinical Nutritional |
| Nutren Junior Liquid | oral | Nestle Clinical Nutritional |
| Nutren Liquid | oral | Nestle Clinical Nutritional |
| NutriSource HN | oral | Novartis Nutritional Corporation |
| Nutrisure Pudding | oral | Abbott Laboratories Ltd |
| Optimental | oral, tube | Abbott Laboratories Ltd |
| Osmolite HN | tube | Abbott Laboratories Ltd |
| PediaSure | oral | Abbott Laboratories Ltd |
| Peptamen | oral | Nestle Clinical Nutritional |
| Peptamen | oral | Nestle Clinical Nutritional |
| Peptinex | oral | Novartis Nutritional Corporation |
| Perative | oral, tube | Abbott Laboratories Ltd |
| Polycose | oral | Abbott Laboratories Ltd |
| Promod | oral, tube | Abbott Laboratories Ltd |
| RCF | oral | Abbott Laboratories Ltd |
| Resource | oral, tube | Novartis Nutritional Corporation |
| SandoSource Peptide | tube | Novartis Nutritional Corporation |
| Subdue Liquid | oral, tube | Novartis Nutritional Corporation |
| Suplena | oral | Abbott Laboratories Ltd |
| Tolerex Powder | tube | Novartis Nutritional Corporation |
| TwoCal HN | oral, tube | Abbott Laboratories Ltd |
| Vital HN Powder | oral | Abbott Laboratories Ltd |
| Vivonex Plus Powder | oral | Novartis Nutritional Corporation |
| Vivonex T.E.N. | oral | Novartis Nutritional Corporation |

### Example 5

The following are examples of dietary formulations of the invention.

Table 5 illustrates an enteral or oral feeding product that contains N-acetyl-D-glucosamine and L-fucose:

TABLE 5
Enteral or Oral Feeding Product

| | Calories from |
|---|---|
| | carbohydrate |
| | component |
| Total Carbohydrate | 100% |
| Carbohydrates one or more of | 97.2% |
| corn syrup solids, sucrose, | |
| fructooligosaccharides, | |
| maltodextrins, lactose, glucose, | |
| and other complex carbohydrates | |
| N-acetyl-D-glucosamine | 2.0% |
| L-fucose | 0.8% |

Table 6 illustrates an enteral or feeding product that contains N-acetyl-D-glucosamine only:

TABLE 6
Enteral or Feeding Product

| | Calories from |
|---|---|
| | carbohydrate |
| | component |
| Total Carbohydrate | 100% |
| Carbohydrates one or more of | 98.0% |
| corn syrup solids, sucrose, | |
| fructooligosaccharides, | |
| maltodextrins, lactose, glucose, | |
| and other complex carbohydrates | |
| N-acetyl-D-glucosamine | 2.0% |
| L-fucose | 0.0% |

Table 7 illustrates an enteral or feeding product that contains L-fucose only:

TABLE 7
Enteral or Feeding Product

| | Calories from |
|---|---|
| | carbohydrate |
| | component |
| Total Carbohydrate | 100% |
| Carbohydrates one or more of | 99.2% |
| corn syrup solids, sucrose, | |
| fructooligosaccharides, | |
| maltodextrins, lactose, glucose, | |
| and other complex carbohydrates | |
| N-acetyl-D-glucosamine | 0.0% |
| L-fucose | 0.8% |

### Example 6

A formula of the invention containing monomeric N-acetyl-D-glucosamine and/or monomeric L-fucose can be prepared by those skilled in the art, with due regard for the nutritional purpose of the product and the carbohydrate component. Examples of such products are illustrated in this Example. In these illustrations N-acetyl-D-glucosamine and L-fucose make up 2.8% by weight of the total carbohydrate present in the product.

Table 8, 9, and 10 are examples of compositions that contain one or both of N-acetyl-D-glucosamine and L-fucose.

TABLE 8
NAG and L-Fucose Composition

| Ingredient | Amount |
|---|---|
| Total Carbohydrates | 100% |
| Glucose | 97.2% of total |
| N-acetyl-D-glucosamine | 2.0% of total |
| L-fucose | 0.8% of total |

TABLE 9
NAG and L-Fucose Composition

| Ingredient | Amount |
|---|---|
| Total Carbohydrate | 100% |
| Glucose | 98.% of total |
| N-acetyl-D-glucosamine | 2.0% of total |
| L-fucose | 0.0% of total |

TABLE 10
NAG and L-Fucose Composition

| Ingredient | Amount |
|---|---|
| Total Carbohydrate | 100% |
| Glucose | 99.2% of total |
| N-acetyl-D-glucosamine | 0.0% of total |
| L-fucose | 0.8% of total |

Comparable amounts of the monomeric N-acetyl-D-glucosamine and or L-fucose could also be added to electrolyte solutions and or the aqueous phase of fat emulsions.

The present invention is not to be limited in scope by the specific embodiments described herein, since such embodiments are intended as but single illustrations of one aspect of the invention and any functionally equivalent embodiments are within the scope of this invention. Indeed, various modifications of the invention in addition to those shown and described herein will become apparent to those skilled in the art from the foregoing description and accompanying drawings. Such modifications are intended to fall within the scope of the appended claims.

All publications, patents and patent applications referred to herein are incorporated by reference in their entirety to the same extent as if each individual publication, patent or patent application was specifically and individually indicated to be incorporated by reference in its entirety. The citation of any reference herein is not an admission that such reference is available as prior art to the instant invention.

1-23. (canceled)

24. A method for enhancing organ development or an immune response, or increasing tissue repair or healing in a subject comprising administering to the subject a therapeutically effective amount of a substantially pure free N-acetyl glucosamine and/or substantially pure free fucose, and optionally substantially pure free sialic acid to provide beneficial effects.

25-38. (canceled)
